SEQUENCE LISTING (8 sequences)

SEQ ID NO: 1 - protein, 381 aa, Human
MPFGNTHNKFKLNYKPEEEYPDLSKHNNHMAKVLTLELYKKLRDKEIPSGFTVDDVIQTGVDNPGHPFIMTVGCVAGDEESYEVFKELFDPIISDRHGGYKPTDKHKTDLNHENLKGGDDLDPNYVLSSPVRTGRSIKGYTLPPHCSRGERRAVEKLSVEALNSLTGEFKGKYYPLKSMTEKEQQQLIDDHFQFDKPVSPLLLASGMARHWPDAPGIWHNDNKSFLVWVNEEDHLRVISMEKGGNMKEVFRRFCVGLQKIEEIFKKAGHPFMWNQHLGYVLTCPSNLGTGLRGGVHVKLAHLSKHPKFEEILTRLRLQKRGTGAVDTAAVGSVFDVSNADRLGSSEVEQVQLVVDGVKLMVEMEKKLEKGQSIDDMIPAQK

SEQ ID NO: 2 - DNA, 1143 bp, Human
atgccattcggtaacacccacaacaagttcaagctgaattacaagcctgaggaggagtaccccgacctcagcaaacataacaaccacatggccaaggtactgacccttgaactctacaagaagctgcgggacaaggagatcccatctggcttcactgtagacgatgtcatccagacaggagtggacaacccaggtcaccccttcatcatgaccgtgggctgcgtggctggtgatgaggagtcctacgaagttttcaaggaactctttgaccccatcatctcggatcgccacgggggctacaaacccactgacaagcacaagactgacctcaaccatgaaaacctcaagggtggagacgacctggaccccaactacgtgctcagcagcccggtccgcactggccgcagcatcaagggctacacgttgcccccacactgctcccgtggcgagcgccgggcggtggagaagctctctgtggaagctctcaacagcctgacgggcgagttcaaagggaagtactaccctctgaagagcatgacggagaaggagcagcagcagctcatcgatgaccacttccagttcgacaagcccgtgtccccgctgctgctggcctcaggcatggcccgccactggcccgacgcccctggcatctggcacaatgacaacaagagcttcctggtgtgggtgaacgaggaggatcacctccgggtcatctccatggagaaggggggcaacatgaaggaggttttccgccgcttctgcgtagggctgcagaagattgaggagatctttaagaaagctggccaccccttcatgtggaaccagcacctgggctacgtgctcacctgcccatccaacctgggcactgggctgcgtggaggcgtgcatgtgaagctggcgcacctgagcaagcaccccaagttcgaggagatcctcacccgcctgcgtctgcagaagaggggtacaggtgcggtggacacagctgccgtgggctcagtatttgacgtgtccaacgctgatcggctgggctcgtccgaagtagaacaggtgcagctggtggtggatggtgtgaagctcatggtggaaatggagaagaagttggagaaaggccagtccatcgacgacatgatccccgcccagaag

SEQ ID NO: 3 - protein, 381 aa, Human
MPFSNSHNALKLRFPAEDEFPDLSAHNNHMAKVLTPELYADVRAKSTPSGFTLDDVIQTGVDNPGHPYIMTVGCVAGDEESYEVFKDLFDPIIEDRHRRYKPSDDDKTDLNPDNLQGGDDLDPNYVLSSRVATGRSIRGFCLPPHCSRGERRAIEKLAVEALSSLDGDLAGRYYALKSMTEAEQQQLIDDHFLFDKPVSPLLLASGMARDWPDAARIWHNDNKTFLVWVNEEDHLRVISMQKGGNMKEVFTRFCTGLTQIETLFKSKDYEFMWNPHLGYILTCPSNLGTGLRAGVDIKLPNLGKHEKFSEVLKRLRLQKRGTGGVDTAAVGGVFDVSNADRLGFSEVELVQMVVDGVKLLIEMEQRLEQGQAIDDLMPAQK

SEQ ID NO: 4 - DNA, 1143 bp, Human
atgcccttctccaacagccacaacgcactgaagctgcgcttcccggccgaggacgagttccccgacctgagcgcccacaacaaccacatggccaaggtgctgacccccgagctgtacgcggacgtgcgcgccaagagcacgccgagcggcttcacgctggacgacgtcatccagacaggcgtggacaacccgggccacccgtacatcatgaccgtgggctgcgtggcgggcgacgaggagtcctacgaagtgttcaaggatctcttcgaccccatcatcgaggaccggcaccggcgctacaagcccagcgatgacgacaagaccgacctcaaccccgacaacctgcagggcggcgacgacctggaccccaactacgtgctgagctcgcgggtggccacgggccgcagcatccgtggcttctgcctccccccgcactgcagccgcggggagcgccgagccatcgagaagctcgcggtggaagccctgtccagcctggacggcgacctggcgggccgatactacgcgctcaagagcatgacggaggcggagcagcagcagctcatcgacgaccacttcctcttcgacaagcccgtgtcgcccctgctgctggcctcgggcatggcccgcgactggcccgacgccgcgcgtatctggcacaatgacaataagaccttcctggtgtgggtcaacgaggaggaccacctgcgggtcatctccatgcagaaggggggcaacatgaaggaggtgttcacccgcttctgcaccggcctcacccagattgaaactctcttcaagtctaaggactatgagttcatgtggaaccctcacctgggctacatcctcacctgcccatccaacctgggcaccgggctgcgggcaggtgtcgatatcaagctgcccaacctgggcaagcatgagaagttctcggaggtgcttaagcggctgcgacttcagaagcgaggcacaggcggtgtggacacggctgcggtgggcggggtcttcgacgtctccaacgctgaccgcctgggcttctcagaggtggagctggtgcagatggtggtggacggagtgaagctgctcatcgagatggaacagcggctggagcagggccaggccatcgacgacctcatgcctgcccagaaa

SEQ ID NO: 5 - DNA, 27 bp, Artificial Sequence
  oligonucleotide designed for PCR primer
ccgaattcatgccattcggtaacaccc

SEQ ID NO: 6 - DNA, 27 bp, Artificial Sequence
  oligonucleotide designed for PCR primer
atggatccctacttctgggcggggatc

SEQ ID NO: 7 - DNA, 27 bp, Artificial Sequence
  oligonucleotide designed for PCR primer
atgaattcatgcccttctccaacagcc

SEQ ID NO: 8 - DNA, 27 bp, Artificial Sequence
  oligonucleotide designed for PCR primer
ccggatcctcatttctgggcaggcatg

(19) United States
(12) Patent Application Publication
(10) Pub. No.: US 2003/0170854 A1
(43) Pub. Date: Sep. 11, 2003

(54) Recombinant human creatine kinase heterodimer with solution-stability

(75) Inventors: Kohji Uchida, Tokyo (JP); Hirokazu Matsukawa, Tokyo (JP); Seiichi Kohda, Tokyo (JP); Osamu Oka, Tokyo (JP); Yuhsi Matuo, Tokyo (JP)

Correspondence Address: NIXON & VANDERHYE, PC, 1100 N GLEBE ROAD, 8TH FLOOR, ARLINGTON, VA, 22201-4714, US

(73) Assignee: Oriental Yeast Co., Ltd.

(21) Appl. No.: 10/412,222

(22) Filed: Apr. 14, 2003

Related U.S. Application Data
(63) Continuation of application No. 09/698,101, filed on Oct. 30, 2000.

(30) Foreign Application Priority Data
Apr. 28, 2000 (JP) 130176/2000

(51) Int. Cl. C12N 9/12; C07H 21/04; C12P 21/02; C12N 5/06

(52) U.S. Cl. 435/194; 435/69.1; 435/320.1; 435/325; 536/23.2

(57) ABSTRACT

A recombinant human creatine kinase heterodimer with solution-stability is disclosed. The CK heterodimer is characterized by being produced by using a vector which contains a nucleic acid encoding the M type subunit of creatine kinase and a nucleic acid encoding the B type subunit thereof.

The present application claims priority based on the Japanese Patent Application No. 130176/2000 filed on Apr. 28, 2000, the entire contents of which are incorporated herein as a reference.

### BACKGROUND OF THE INVENTION

The present invention is directed to providing a creatine kinase heterodimer recombinant protein which remains stable when dissolved in solutions.

Creatine kinase (which will be referred simply as CK or CPK in some cases) is an enzyme which catalyzes transphosphorylation from ATP to creatine, as shown below:

ATP+creatine→ADP+phosphocreatine.

This enzyme can be found in wide variety of animals. In particular, it is contained in a large amount in tissues which consume a large amount of energy over a short period of time, for example, white muscle; cardiac muscle; brain; and sperm of vertebrates. In diagnosing various physiological conditions it is of great importance to assay the enzymatic activity level of CK. An increase in CK level is considered to be closely related to clinical conditions of, in particular, myocardial infarction, myocardial ischemia, angina pectoris, pulse frequens, myocarditis, subarachnoidal bleeding, apoplexy, brain tumor, meningitis, encephalitis, and so on.

There are known three CK isozymes (MM, MB and BB types). Due to the human organ specificity of each isozyme, it has been a general practice, for example, when conducting clinical diagnosis of heart diseases such as myocardial infarction to assay the particular CK type which infiltrates into the blood from the cardiac muscle. In particular, it is expected that specific and definite diagnostic data can be obtained at the earliest stage by assaying the CK-MB type. Thus, various methods for assaying CK-MB have been developed. For example, ion exchange chromatography, electrophoresis, antibody inhibitory activity assay and immune analysis are described in “Rinsho Kensa-ho Teiyo (Clinical Examination Handbook)” (31st revised version), pp. 640-643, written by Izumi Kanai, edited by Masamitsu Kanai, Kanahara Shuppan (1998).

When employing the above-noted assaying methods, it is necessary to use purified CK, in particular CK-MB, as both an analytical standard and a quality control substance. In the case of attempting to purify CK-MB from a natural material by ion-exchange chromatography, gel filtration chromatography and/or affinity column chromatography, however, it is known that creatine kinase existing in the serum also contains subfractions formed by limited proteolysis, etc. Namely, it is highly difficult to prepare a pure specimen from pooled serum. Moreover, organs, particular human organs are available from only limited amount of sources, and CK can be prepared only in trace amounts from cultured cells. Accordingly, the development of a method by which a CK standard can be easily produced on a mass scale has been in urgent demand.

It has been pointed out that with respect to CK-MB in particular, a problem of stability exists. For example, Steghers J. P. et al. (Clin. Chem., vol. 29, p. 1537, 1983) reported that CK-MB activity in human blood is lowered by 22% after being stored at 4° C. for 4 days. Therefore, attempts have been made to stabilize human CK having such poor stability in solutions by adding various stabilizers. Examples of these attempts include stabilization by converting to reducing sugar(s) (Japanese Patent Public Disclosure: No.253378/87), stabilization by adding reduced glutathione (Japanese Patent Public Disclosure: No.252797/97); stabilization by reacting with disulfide and/or thiosulfonate (Japanese Patent Public Disclosure: No.118889/87) and stabilization by using a non-thiol reducing agent (Japanese Patent Public Disclosure: NO.189760/94). Further, Lavy et al. (Clin Chem., vol. 21, No. 11, p. 1691, 1975) report a method of reconstituting MB type creatine kinase in vitro from MM and BB types obtained from a natural source. Considering the complicated procedures required and also the effects on other serum components, however, these additives and stabilization methods as described above give rise to problems in practice.

On the other hand, attempts have also been made to produce recombinant CK proteins by using genetic engineering techniques. Concerning the production of recombinant human CK, for example, it is reported that CK-BB was expressed in insect cells (De Kok Y J M et al., Mol. Cell Biochem., 1995, vol. 143, No. 1, pp. 59-65). The Domestic Announcement No. 504698/97 of the PCT Inter-national Public Disclosure W095/12662 describes a method of preparing a heterodimer (MB type) by cotransfecting a procaryotic host (for example, Escherichia coil) with a first vector carrying a gene encoding the M type or B type subunit inserted thereinto, and a second vector carrying a gene encoding the B type or M type subunit inserted thereinto. By the method of preparing CK via the cotransfection with the use of the first and second vectors, three isozymes, namely, CK-MB, CK-MM and CK-BB are formed. However, the ratio of CK-MB in relation to total CK activity expressed per transformant is neither discussed nor stated in the Domestic Announcement No. 504698/97. To economically utilize CK-MB in the clinical field on an industrial scale, it is necessary to effect a method whereby recombinant CK-MB can be both preferentially and efficiently obtained.

Japanese Patent Public Disclosure: No.292585/94 describes a method for preparing a typical creatine kinase isoform. More particularly, genes encoding the M type or B type subunits are subjected to site-specific mutation by polymerase chain reaction (PCR) so that the C-end lysine residue is deleted from the M type or B type subunits. In this reference, use is made of a procedure whereby a host is cotransfected with two distinct vectors containing genes encoding these subunits, respectively, to thereby express three isozymes, similar to the Domestic Announcement No. 504698/97. In the Japanese Patent Public Disclosure: No.292585/94, furthermore, it is required to perform mutation whereby the C-end lysine residue is deleted. Although it cannot be judged whether or not such a deleted mutant sustains the same physicochemical, enzymological and immunochemical characteristics as the natural CK, no discussion is made with regard to this issue in the said reference. Namely, the recombinant CK thus obtained was merely measured with the stability of the activity and was separated by electrophoresis.

Accordingly, prior to the present invention, there has been no established method by which a CK-MB heterodimer at a high yield could be obtained easily.

### SUMMARY OF THE INVENTION

An object of the present invention is to provide a vector containing a nucleic acid encoding the M type subunit of creatine kinase and a nucleic acid encoding the B type subunit thereof. In the vector according to the present invention,

i) the M type subunit is a polypeptide having the amino acid residues 1 to 381 in SEQ ID NO:1, or a polypeptide having an amino acid sequence derived from the above sequence by deletion, substitution or addition of one or more amino acid residues and having enzymatic activity; and

ii) the B type subunit is a polypeptide having the amino acid residues 1 to 381 in SEQ ID NO:3, or a polypeptide having an amino acid sequence derived from the above sequence by deletion, substitution or addition of one or more amino acid residues and having enzymatic activity.

Another object of the present invention is to provide a host cell transformed by said vector.

A further object of the present invention is to provide a method of producing a creatine kinase heterodimer recombinant protein containing the M type subunit and the B type subunit of creatine kinase.

Still another object of the present invention is to provide a creatine kinase heterodimer recombinant protein containing the M type subunit and the B type subunit of creatine kinase wherein said recombinant protein is produced by the method as described above.

A still further another object of the present invention is to provide a solution-stable composition comprising a creatine kinase heterodimer recombinant protein containing the M type subunit and the B type subunit of creatine kinase.

### BRIEF DESCRIPTION OF THE DRAWINGS

FIG. 1 is a diagram showing the construction process of a vector containing a nucleic acid encoding the M type subunit of CK and a nucleic acid encoding the B type subunit of CK according to the present invention.

FIG. 2 shows the separation of a recombinant human CK-MB heterodimer by using a Sepharose column.

FIG. 3 is a graph showing changes with the passage of time in the residual activity of a composition containing the recombinant human CK-MB heterodimer according to the present invention stored in a refrigerated state.

### DETAILED DESCRIPTION OF THE INVENTION

The present inventors conducted intensive studies to solve the above-described problems. As a result, they have successfully obtained a recombinant CK-MB heterodimer at a high yield by ligating a gene encoding the M type subunit to a gene encoding the B type subunit and inserting it into a single vector, thereby completing the present invention.

M Type and B Type Subunits of CK

The vector according to the present invention is characterized by having both a gene encoding the M type subunit of creatine kinase and a gene encoding the B type subunit thereof.

The amino acid sequences of the M type subunit and the B type subunit of CK are publicly known and presented in, for example, Perryman, M. B. et al., Biochem. Biophys. Res. Commun., 140(3), pp. 981-989 (1986); and Villarreal-Levy, G. et al., Biochem. Biophys. Res. Commun., 144 (3) pp. 1116-1127 (1987). Typically, the CK-M subunit of the present invention has amino acid sequences comprising the 381 amino acids represented by SEQ ID NO:1 in the Sequence Listing. SEQ ID NO:1 is a human amino acid sequence (Genbank Reg. No. NM 0001824, Perryman, M. B. et al., 19-MAR-1999). Typically, the CK-B subunit of the present invention has amino acid sequences comprising the 381 amino acids represented by SEQ ID NO:3 in the Sequence Listing. SEQ ID NO:3 is a human amino acid sequence (Genbank Reg. No. NM 0001823, Villarreal-Levy, G. et al., 19Mar. 1999).

It is well known that natural proteins have variants having one or more amino acid mutations caused by mutations in genes due to differences in the varieties of species producing the same or differences in ecological systems, or by the occurrence of closely similar isozymes. In addition to the amino acid sequences of SEQ ID NOS:1 and 3 which are respectively the amino acid sequences of human CK-M and CK-B subunits, use can be made in the present invention of those originating in nonhuman animals belonging to the primates and other mammals (bovine, mouse, sheep, canine, etc.). Therefore, the CK-M subunit according to the present invention includes a polypeptide having the amino acid residues 1 to 381 in SEQ ID NO:1 and polypeptides having an amino acid sequence derived from this sequence by mutation such as deletion, substitution or addition of one or more amino acid residues and having (exhibiting) enzymatic activity. Similarly, the CK-B subunit according to the present invention includes a polypeptide having the amino acid residues 1 to 381 in SEQ ID NO:3 and polypeptides having an amino acid sequence derived from this sequence by mutation such as deletion, substitution or addition of one or more amino acid residues and having enzymatic activity.

The term “amino acid mutation” as used herein means deletion, substitution, insertion and/or addition of one or more amino acids. Although the CK-M type subunit and the CK-B type subunit according to the present invention typically have the amino acid sequences represented by SEQ ID NOS:1 and 3, respectively, these subunits are not restricted thereto, and can include any homologous proteins so long as such proteins have the characteristics as described herein. The homology percentage is at least 70%, preferably at least 80% and still preferably at least 90%.

In the present invention, the homology percentage can be determined by comparing the sequence data by using, for example, a BLAST program reported by Altschul et al. (Nucl. Acids. Res., 25, pp. 3389-3402, 1997). This program is available from the Internet Wen Site of National Center for Biotechnology Information (NCBI) or DNA Data Bank of Japan (DDBJ). Various conditions (parameters) for searching the homology by using the BLAST program are presented in detail in the above sites. Although some parts of the configuration can be optionally altered, searches can be performed usually by employing the defaults.

In general, a mutant protein obtained by substituting one or more amino acid residue by other one or more amino acid residue having similar properties (for example, substitution of a hydrophobic amino acid by another hydrophobic amino acid, substitution of a hydrophilic amino acid by another hydrophilic amino acid, substitution of an acidic amino acid by another acidic amino acid, or substitution of a basic amino acid by another basic amino acid) has properties similar to the intact protein. Procedures for preparing recombinant proteins having such a desired mutation by using genetic engineering techniques are well known to persons skilled in the art and, therefore, these mutant proteins also fall within the scope of the present invention.

In another example, a sequence encoding a Cys residue is modified in such a manner as to induce the deletion of the Cys residue or the substitution thereof by another amino acid so that the formation of an inappropriate intramolecular disulfide cross-link during the step of regeneration can be inhibited. The substituent amino acid is selected from among tryptophan, serine, aspartic acid and lysine, with tryptophan being the most desirable.

It is also possible to delete or add an amino acid sequence by considering the potential effect on the biological activity (enzymatic activity) by deletion or insertion. In the case where an analog having reduced carbohydrates is expressed by using a yeast expression system, for example, glycosylation can be avoided by modifying the N-glycosylation site. The glycosylation site in a eucaryotic polypeptide is characterized by the amino acid triplet Asn-X-Y, wherein X is an arbitrary amino acid other than Pro, and Y is Ser or Thr. By appropriately modifying a nucleotide sequence encoding this triplet, it is expected that substitution, addition or deletion will result which would inhibit the bonding of a carbohydrate residue to the Asn side chain. It is also possible to enhance the expression in a yeast system showing KEX2 protease activity by modifying a sequence encoding a dibasic amino acid residue.

It is preferable for the CK-B type subunit and the CK-M type subunit according to the present invention to be able to sustain at least one of the physicochemical properties of the corresponding natural subunit. The term “enzymatically active” as used herein means that the CK-B subunit and the CK-M subunit can form a homodimer or a heterodimer and exert activity as creatine kinase similar to natural subunits, even though the CK-B and CK-M subunits have amino acid sequences which are different from SEQ ID NOS:1 and 3 respectively.

CK-M Type Subunit and CK-B Type Subunit Genes

As will be described in Examples hereinafter, the CK heterodimer according to the present invention can be expressed by genetic engineering using a CK-M type subunit gene and a CK-B type subunit gene. The genes encoding the CK-M type subunit and CK-B type subunit of the present invention may be arbitrarily selected from among natural DNAs, recombinant DNAs and chemically synthesized DNAs without any restriction. Moreover, either genomic DNA clones or cDNA clones may be used therefor. The genes encoding the CK-M type subunit and CK-B type subunit of the present invention can be easily obtained by a person skilled in the art by using genetic engineering techniques which are described herein or which have been commonly employed in the art.

Typically, the CK-M type subunit gene has the nucleotide sequence 1-1143 described in SEQ ID NO:2 in the Sequence Listing (Genbank Reg. No. NM 0001824). Typically, the CK-B type subunit gene has the nucleotide sequence 1-1143 described in SEQ ID NO:4 in the Sequence Listing (Genbank Reg. No. NM 0001823). SEQ ID NOS:2 and 4 show the nucleotide sequences of genes encoding the human M-and B-subunits respectively. However, it is well known to a person skilled in the art that variants in natural proteins arise due to the existence of mutations caused in which a number of mutations may exist due to the occurrence of species differences in the same or differing ecological systems, or due to the occurrence of closely similar isozymes. Accordingly, the genes of the CK-M and CK-B subunits of the present invention are not restricted to those genes having the nucleotide sequences of SEQ ID NOS:2 and 4 provided in the Sequence Listing, but may include any genes encoding the polypeptides of the CK-M and CK-B subunits as described above.

The genes of the CK-M and CK-B subunits of the present invention having the human gene nucleotide sequences of SEQ ID NOS:2 and 4 and those having nucleotide sequences other than SEQ ID NOS:2 and 4 can be both obtained by using basic techniques in genetic engineering such as hybridization or nucleic acid amplification on the basis of the amino acid sequences of the human CK-M and CK-B subunit proteins as represented by SEQ ID NOS:1 and 3 in the Sequence Listing and DNA sequences encoding the same or parts thereof. It is also possible to isolate a gene having similar physiological activity from humans or other species by using such genetic engineering techniques.

Screening of a gene may be carried out under arbitrary conditions without any restriction. In general, it is preferable to employ stringent conditions (for example, 6×SSC, 5×Denhard's, 0.1% SDS, 25 to 68° C.). In such a case, the hybridization temperature is controlled still preferably at 45 to 68° C. (without formamide) or 25 to 50° C. (with 50% formamide). It is well known to a person skilled in the art that DNAs which contain nucleotide sequences having a homology of a certain level or above can be cloned by appropriately setting the hybridization conditions (formamide concentration, salt concentration, temperature, etc.). Homologous genes thus cloned are also usable in producing the recombinant CK heterodimer according to the present invention.

Examples of the nucleic acid amplification reactions include reactions which are carried out utilizing temperature circulation such as polymerase chain reaction (PCR) (Saiki et al., 1985, Science 230, pp. 1350-1354), ligase chain reaction (LCR) (Woh et al., 1989, Genomics 4, pp. 560-569; Baringer et al., 1990, Gene 89, pp. 117-122; and Baranny et al., 1991, Proc. Natl. Acad. Sci. USA 88, pp. 189-193) and amplification based on transcription (Kwoh et al., 1989, Proc. Natl. Acad. Sci. USA 86, pp. 1173-1177) and isothermal reactions such as strand displacement amplification (SDA) (Walker et al., 1992, Proc. Natl. Acad. Sci. USA 89, pp. 392-396; and Walker et al., 1992, Nuc. Acids. Res. 20, pp. 1691-1696), self-sustained sequence replication (3SR) (Guatelli et al., 1990, Proc. Natl. Acad. Sci. USA 87, pp. 1874-1878) and Qβ replicase system (Lizardi et al., 1988, BioTechnology 6, pp. 1197-1202). Moreover, use can be made of a nucleic acid sequence based amplification (NASABA) on the basis of competitive amplification of a target nucleic acid and a mutant sequence as reported in European Patent No. 0525882 and the like. It is preferable to use the PCR method therefor.

Such a homogeneous gene cloned by using the above-described hybridization, nucleic acid amplification, etc. has a homology of at least 70%, preferably at least 80% and still preferably at least 90% with the nucleic acid sequence represented by SEQ ID NO:2 or 4.

Oligonucleotide primers to be used in the nucleic acid amplification for obtaining the genes of the CK-M and CK-B type subunits can be constructed based on the nucleotide sequences of the genes encoding the CK-M and CK-B type subunits. More particularly, these oligonucleotides can be constructed by, for example, selecting two domains satisfying the following requirements from the nucleotide sequence of the gene encoding the human CK-M or CK-B type subunit represented by SEQ ID NO:2 or 4;

1) each domain consists of 15 to 30 bases;

2) each domain has 40% to 60% of G+C; and

3) the distance between these domains is from about 100 to 1000 bases;

preparing single-stranded DNAs having nucleotide sequences which are identical to the nucleotide sequences of the above domains or complementary thereto, or preparing a single-stranded DNA mixture having degeneracy in the genetic code which ensures that the amino acid residues encoded by the above-described single-stranded DNAs are not changed; and optionally modifying the single-stranded DNAs while avoiding damage to the binding specificity to the nucleotide sequences of the genes encoding the above proteins. These oligonucleotides can be used in the hybridization to detector isolate the genes of the CK-M and CK-B type subunits according to the present invention. It is also possible to use an appropriate pair of these oligonucleotides in the amplification reactions such as PCR.

Similarly, genes of the CK-M and CK-B type subunits according to the present invention which have nucleotide sequences other than the human genes represented by SEQ ID NOS:2 and 4 can be obtained by, for example, site-directed mutagenesis (see, for example, Nucleic Acid Research, Vol. 10, No. 20, pp. 6487-6500, 1982) with the use of the amino acid sequences of the human CK-M and CK-B type subunits represented by SEQ ID NOS: 1 and 3 as presented herein, DNA sequences encoding these amino acid sequences or parts thereof.

The site-directed mutagenesis can be carried out in the following manner by using, for example, a synthetic oligonucleotide primer which is complementary to the single-stranded DNA to be mutagenized (for example, a phage) other than a definite disagreement, i.e., the desired mutation. By using this synthetic oligonucleotide as a primer, a strand complementary to the above-described single-stranded DNA is synthesized, and host cells are transformed by the double-stranded DNA thus obtained. The transformed host cells are transferred onto an agar plate and plaques are formed from individual cells. Theoretically, 50% of the new colonies thus formed have the DNA carrying the desired mutation while the remaining 50% have the intact sequence. Thus, the obtained plaques are hybridized with a synthetic probe labeled with a radioisotope, etc. at a temperature at which plaques having DNA completely agreeing with the DNA having the above-described desired mutation are hybridizable but plaques not agreeing therewith (i.e., those having the intact sequence) are not hybridizable. Subsequently, the hybridized plaques are collected and incubated and the DNA is then recovered.

Vector for Producing CK Heterodimer

A characteristic of the present invention resides in that a gene encoding the M type subunit and the gene encoding the B type subunit are ligated together in tandem in a single vector to produce the recombinant CK heterodimer.

The genes respectively encoding these subunits may be consecutively bonded to each other in-frame. Alternatively, a DNA encoding a linker sequence may be located between these genes, though the present invention is not restricted thereto. Although the linker sequence is not particularly restricted, it consists preferably of 6 to 20 bases. More particularly, use can be made as the linker of a sequence consisting of repeated sequences of amino acid residues in tandem such as (GGGGS)ₙ wherein n is preferably 3. Alternatively, commercially available linker sequences (for example, Linker Primer Mix manufactured by Pharmacia Biotech) may be employed. Owing to the presence of the linker peptide, the M type subunit and the B type subunit exist in proximity to each other at a ratio of 1:1, which facilitates the formation of the CK-MB heterodimer.

Alternatively, a promoter, a Shine-Dalgarno (SD) sequence, which participates in the formation of an initiation complex by the complementary base association with 16S rRNA in the biosynthesis of a protein, etc. may be located between the genes respectively encoding the subunits. When these subunits are governed by a single promoter, mRNAs respectively encoding the CK-M and CK-B type subunits are transcribed at the same ratio. When these subunits are governed individually by different promoters, the transcription dose of the mRNA of each subunit can be controlled. In either case, the mRNA transcriptional ratio between the subunits can be controlled irrespective of the proliferation ability of the vector by inserting the genes respectively encoding the subunits into a single vector together with promoter(s).

Ligation of the M type subunit and the B type subunit in tandem with the use of the genes according to the present invention enables the mass expression. Since the M type subunit and the B type subunit are expressed by the same vector, the subunits thus transcribed and translated are located closely to each other and thus the MB heterodimer can be easily formed in a stable state, as compared with the conventional case where genes respectively encoding the subunits are inserted into different vectors and then cotransfected. As a result of insertion into a single vector, in addition, the mRNA transcription ratio between the subunits can be easily controlled by, for example, regulating promoters, which further facilitates the formation of the MB heterodimer.

The DNA fragments of the present invention may be integrated into a vector such as a plasmid in accordance with, for example, a method reported by Sambrook, J. et al., Molecular Cloning, A Laboratory Manual, (second edition), Cold Spring Harbor Laboratory, 1.53 (1989). A commercially available ligation kit can be used (for example, a product manufactured by Takara Shuzo). The recombinant vector thus obtained (for example, a recombinant plasmid) is transferred into host cells (for example, E coli. JM109, TB1, LE392 or XL-lBlue).

Examples of the method of transferring a plasmid into host cells include the calcium phosphate method or the calcium chloride/rubidium chloride method, electroporation, electroinjection, treatment with a chemical, such as PEG, the method of using gene shotgun, etc. as described in Sambrook, J. et al., Molecular Cloning, A Laboratory Manual, (second edition), Cold Spring Harbor Laboratory, 1.74 (1989).

The vector can be prepared by ligating the desired genes to a recombinant vector (for example, a plasmid DNA) available in the art. Specific examples of the vector usable herein include plasmids originated from E. coli such as pBluescript, pUC18, pUC19 and pB322, though the present invention is not restricted thereto.

To produce the desired protein, an expression vector is particularly useful. The expression vector is not restricted in type, so long as it has a function of expressing the desired genes in various procaryotic and/or eucaryotic host cells and thus producing the desired protein. Preferred examples of the vector include expression vectors for E. Coli such as pQE-30, pQE-60, pMAL-C2, pMAL-p2 and pSE420; expression vectors for yeasts such as pYES2 (the genus of Saccharomyces) and pPIC3.5K, pPIC9K and pAO815 (the genus of Pichia); and expression vectors for insects such as pBacPAK8/9, pBK283, pVL1392 and pBlueBac4.5. Preferably, pTRP, which is an expression vector for E. Coli, may be used (Clin. Chim. Acta., 1995, Jun. 15;237 (1-2):43-58).

A transformant can be prepared by transferring a desired expression vector into a host cell. The host cell to be used herein is not particularly restricted, but can be arbitrarily selected from among various cells commonly employed in the art including natural cells and artificially established recombinant cells, so long as said cell is compatible with the expression vector of the present invention and can be transformed thereby. Examples of a host cell include bacteria belonging to the genera Escherichia, Bacillus, etc., yeasts belonging to the genera Saccharomyces, Pichia, etc., animal cells, insect cells and plant cells.

Preferably, E. coli, yeasts or insect cells can be used as the host cell. Particular examples thereof include E. coli strains (M15, JM109, BL21, etc.), yeasts (INVScl (Saccharomyces), GS115, KM71 (each Pichia)), and insect cells (BmN4, silkworm larva, etc.). Examples of animal cells include cells originating in mouse, Xenopus, rat, hamster, monkey and humans and cultured cell lines established from the above cells. It is particularly preferable to use E. coli, still preferably E. coli JM109 (available from, for example, Takara Shuzo) as the host cell.

In the case of using a bacterium (in particular E. coli) as the host cell, the expression vector generally consists of at least of a promoter/operator domain, an initiation codon, a gene encoding the CK-M type subunit, a gene encoding the CK-B type subunit, a termination codon, a terminator and a replicable unit.

In the case of using a yeast, a plant cell, an animal cell or an insect cell as the host cell, it is generally preferable for the expression vector to contain at least a promoter, an initiation codon, a gene encoding the CK-M type subunit, a gene encoding the CK-B type subunit, a termination codon and a terminator. Moreover, said expression vector may optionally contain a DNA encoding a signal peptide, an enhancer sequence, the non-translation domains in the 5′- and 3′-sides of a desired gene, a selection marker domain, a replicable unit and the like.

An appropriate example of the initiation codon in the vector according to the present invention is methionine codon (ATG). Examples of the termination codon include termination codons commonly employed such as TAG, TGA and TAA.

The term “replicable unit” as used herein means a DNA capable of replicating its entire DNA sequence in a host cell. Examples thereof include natural plasmids, artificially modified plasmids (i.e., plasmids prepared from natural plasmids) and synthetic plasmids. Preferable examples of the plasmid include plasmids pQE3, pET, pCAL or artificial modifications thereof (e.g. DNA fragments obtained by treating pQE30, pET or pCAL with appropriate restriction enzyme(s)) in case of E. coli; plasmids pYES2 and pPIC9K in case of yeasts; and a plasmid pBacPAK8/9, etc. in case of insect cells.

As the enhancer sequence and the terminator sequence, use may be made of those commonly employed in the art, for example, sequences originating in SV40.

As the selection marker, use may be made of those commonly employed in the art by using a common method. Examples thereof include antibiotic resistance genes (tetracycline, ampicillin, kanamycin, neomycin, hygromycin, spectinomycin, etc.).

The expression vector can be prepared by ligating the above-described promoter, the initiation codon, the gene encoding the CK-M type subunit, the gene encoding the CK-B type subunit, the termination codon and the terminator domain, consecutively and cyclically to an adequate replicable unit. In this process, it is also possible to use adequate DNA fragment(s) (linker, other restriction enzyme sites, etc.) by a common method such as digestion with restriction enzyme(s) or ligation with the use of T4DNA ligase, if desired.

The CK-MB heterodimer according to the present invention may be bonded to a gene encoding a protein or a polypeptide so that it is expressed in a covalently bonded or aggregated state with the protein or polypeptide. An example of such fusion contains a signal or leader polypeptide (for example, Saccharomyces α factor leader) in the N- or C-end domain of a recombinant protein which participates in the transfer of a complex from the synthesis site to the inside or outside of a cell membrane or cell wall simultaneously with or after the completion of the translation. Alternatively, use may be made of a CK-MB heterodimer fusion containing a polypeptide (for example, poly-His) which is added to facilitate the purification and identification of the CK-MB heterodimer.

Such a fused protein can be prepared by using the conventional technique of cleaving a fragment from a desired sequence with an enzyme and ligating. The PCR technique employing synthetic oligonucleotides is usable in preparing and/or amplifying a desired fragment. Also, synthetic oligonucleotides showing desired sequences can be used in preparing a DNA construction encoding the fused protein. The fused protein may contain one or more additional sequences such as leader (or signal peptide) sequences, oigomerization domain (for example, leucine zipper or other adequate zippers) linker sequences, and sequences encoding a portion with a high immunogenicity capable of providing a means for easily purifying or quickly detecting the fused protein.

A signal peptide facilitates the secretion of a protein from cells. Flag® Octapeptide (Hopp et al., Bio/Technology, 6:1204, 1988) provides an epitope reversibly bonded to a specific monoclonal antibody, with said epitope having high immunogenicity while not having any effect on the biological activity of the fused protein, thereby enabling rapid detection and convenient purification of the fused protein expressed. The Flag® sequence is cleaved specifically by bovine mucosal enterokinase at the residue immediately following the Asp-Lys pair and the fused protein capped by the peptide this sequence is tolerant to intracellular digestion in E. coli. A murine monoclonal antibody binding to Flag® has been deposited with ATCC (Accession No. HB 9259), while a method of purifying a fused protein containing the Flag® sequence by using an antibody is described in U.S. Pat. No. 5,011,912 which is cited incorporated herein as a reference.

The genes encoding the CK-M type subunit and/or the CK-B type subunit according to the present invention may be bonded to and expressed with a gene encoding the constant region (hereinafter referred to as the Fc region). An adequate Fc region is capable of binding to protein A or protein G or can be recognized by an antibody which is usable in purifying or detecting a fused protein containing the Fc region. As the Fc region, use can be made of a publicly known human IgG1 Fc region or murine IgG1 Fc region. It is also possible to use an adequate fragment of Fc region, for example, a human IgG1 Fc region fragment from which the amino acid sequence responding to the binding to protein A has been deleted so that the fragment is capable of binding to protein G but not to protein A.

pTRP-hCKMB as will be described in Example 1 is usable as the recombinant expression vector according to the present invention, though the present invention is not restricted thereto. This pTRP-hCKMB comprises a gene encoding the CK-M type subunit and a gene encoding the CK-B type subunit which are ligated via a pTRP promoter sequence and a SD sequence. The gene encoding the CK-M type subunit and the gene encoding the CK-B type subunit are controlled in expression respectively by the pTRP promoter located upstream. pTRP-hCKMB further contains another constitution element, such as ori for the replication in E. coli host cells, Amp as a selection marker for screening a transformant.

The expression vector of the present invention can be introduced (i.e., transformation (transduction)) into a host cell by publicly known methods.

Namely, the transformation can be carried out by, for example, a method reported by Cohen et al. (Proc. Natl. Acad. Sci. USA, 69, 2110 (1972))), the protoplast method (Mol. Gen. Genet., 168, 111 (1979)) or the competent method (J. Mol. Biol., 56, 209 (1971) in case of bacteria (E. coli, Bacillus subtilis, etc.); a method reported by Hinnens et al. (Proc. Natl. Acad. Sci. USA, 75, 1927 (1978)) and the lithium method (J. Bacteriol., 153, 163 (1988)) in case of Saccharomyces cerevisiae; the leaf disc method (Science, 227, 129 (1985)) and the electroporation method (Nature, 319, 791 (1986)) in case of plant cells; a method reported by Graham (Virology, 52, 456 (1973)) in the case of animal cells; and a method reported by Summers et al. (Mol. Cell. Biol., 3, 2156-2165 (1983)) in the case of insect cells.

In Examples of the present invention, a gene encoding the CK-M type subunit and a gene encoding the CK-B type subunit are ligated in tandem and inserted into the above-described expression vector pTRP for E. coli. Then E. coli JM109 strain (manufactured by Takara Shuzo) was transformed thereby as the host cell. The transformant JM109/pTRP-hCKMB thus obtained was deposited with National Institute of Bioscience and Human-Technology, Agency of Industrial Science and Technology, MITI (1-1-3, Higashi, Tsukuba-shi, Ibaragi-ken, 305-0046) under the accession number FERM BP-7141 on Apr. 20, 2000.

Expression and Purification of CK Heterodimer Protein

The thus constructed vector, which contains the nucleic acid encoding the M type subunit of creatine kinase and the nucleic acid encoding the B type subunit thereof ligated in tandem, is transformed into an adequate host cell. Thus, the CK heterodimer protein according to the present invention can be produced. More particularly, the host cells transformed by the vector of the present invention are cultured under conditions allowing the expression of a recombinant protein and the CK heterodimer recombinant protein containing the M type subunit and the B type subunit of creatine kinase is recovered form the culture medium.

The conditions appropriate for the expression of the recombinant protein are, for example, as follows, though the present invention is not restricted thereto. In the case where E. coli is employed as the host, the host cells are incubated in LB medium (0.5% of yeast extract, 1% of polypeptone, 1% of NaCl) at 25 to 42° C., preferably at 37° C., for 8 to 16 hours.

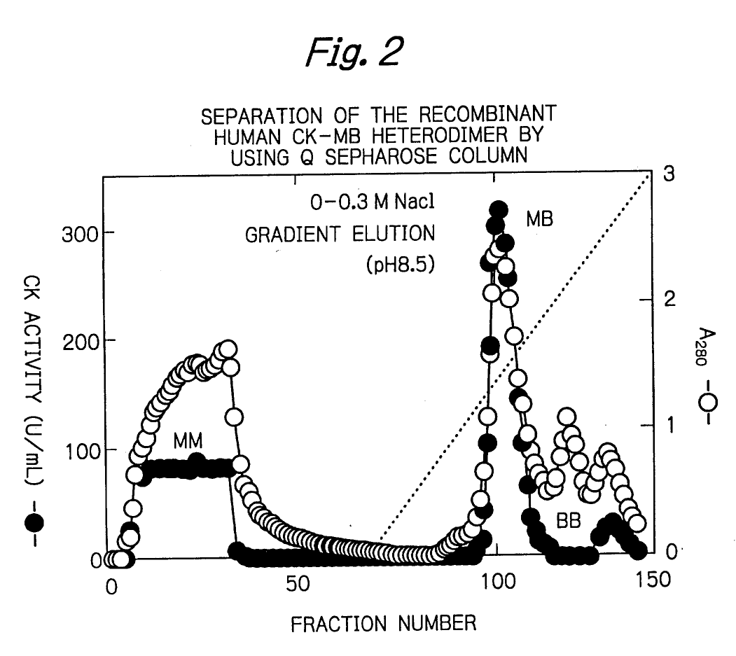

The recombinant CK heterodimer expressed by genetic engineering can be purified by purification techniques commonly employed in the art, for example, ammonium sulfate precipitation, gel filtration column chromatography, etc. More particularly, a CK activity fraction can be obtained by optionally treating the host cells ultrasonically and then subjecting the cells to an appropriate combination of techniques which have been commonly employed for purifying and isolating proteins, for example, ammonium sulfate precipitation, gel filtration column chromatography (Buthyl-Toyopearl C650 gel manufactured by Tosoh, Superdex Pg75 manufactured by Pharmacia, etc.) and the like. By using a Sepharose column such as a Q-Sepharose column (manufactured by Pharmacia, Sweden), the CK fraction can be further divided into MM-, MB- and BB-peaks (Example 2, FIG. 2). Moreover, the purification may be further performed by using a Sephacryl-S200HR column (manufactured by Pharmacia, Sweden), etc. Thus the CK-MB fraction can be separated from the fraction showing the CK activity (Example 2).

The CK activity of the CK heterodimer can be confirmed. A method for assaying human CK activity is described in detail in Rinsho Kagaku (Clinical Chemistry), 1990, vol. 19:189 “Recommended Method for Assaying Enzyme Activity in Human Serum-Creatine Kinase” and a method for preparing assay reagents and assay procedures are stated in “Rinsho Kensa-ho Teiyo (Clinical Examination Handbook)” (31st revised version), pp. 636-640, written by Izumi Kanai, edited by Masamitsu Kanai, Kanahara Shuppan (1998) as cited above. It is also possible to assay human CK activity by using commercially available clinical test reagents for assaying CK activity in human serum. Examples of such commercially available products include Merck Autoliquid CK (manufactured by Kanto Chemical). As an analytical instrument for assaying human CK activity, a spectrophotometer can be used. Alternatively, the CK activity can be assayed with an automated analyzer for clinical test (for example, Hitachi Model 71500 manufactured by Hitachi) by using commercially available CK assay reagents for clinical testing in accordance with the manufacturer's instructions.

Composition Containing CK Heterodimer

The present invention further provides compositions containing the CK heterodimer recombinant protein comprising CK-M and CK-B.

Prior to the present invention, it has been the practice to use stabilizers such as reducing sugars, reduced glutathione, disulfide and/or thiosulfonate and non-thiol reducing agents in order to stabilize CK. When dissolved in a solvent (for example, serum, plasma, or artificial serum prepared by adding albumin to physiological saline to give a concentration (e.g., 0.5% w/v) comparable to serum level) under stabilizer-free conditions, the composition according to the present invention shows a solution stability of at least 80%, preferably at least 85%, after being stored in a refrigerated state (11° C. or lower, preferably 2 to 8° C.) for 4 months. That is to say, the composition comprising the CK heterodimer according to the present invention can be stably stored over a long period of time as a solution and, therefore, can be conveniently employed without the need to dissolve a freeze-dried powder before each use.

The composition according to the present invention is characterized by containing the recombinant heterodimer obtained by the production method as described above. A method of assaying human serum CK and/or CK isozymes is described in “Rinsho Kensa-ho Teiyo (Clinical Examination Handbook)” (31st revised version), pp. 636-643, written by Izumi Kanai, edited by Masamitsu Kanai, Kanahara Shuppan (1998). As stated in this document, it is preferable to use a composition comprising a recombinant CK heterodimer as a control for assaying human CK-MB. To assay the total human CK activity, use can be also made of a recombinant human CK-MB heterodimer composition and/or a composition containing a recombinant CK isozyme mixture containing a recombinant human CK-MB heterodimer and CK-MM and/or CK-BB homodimers as an assay control. The composition according to the present invention includes within its scope a recombinant human CK-MB heterodimer composition and a recombinant human CK composition comprising an isozyme mixture.

The CK heterodimer of the present invention can be formulated into preparations sustaining its activity and utilized as drugs, preferably controls in clinical diagnosis and quality control substances which are to be used to confirm that routine clinical examinations are exactly and accurately conducted. For these purposes, a CK heterodimer of the present invention can be produced on a mass scale by using the genes encoding the CK-M and CK-B type subunits which are ligated in tandem in a single vector.

The CK heterodimer-containing the composition of the present invention are usable in diagnosing physiological conditions in association with an increase in CK levels, namely, in diagnosing, preventing and treating clinical conditions such as myocardial infarction, myocardial ischemia, angina pectoris, pulse ferquens, myocarditis, subarachnoidal bleeding, apoplexy, brain tumor, meningitis, encephalitis, an so on.

For use as a control in clinical diagnosis or a quality control substance for routine clinical examinations, the recombinant human CK-MB heterodimer is added at a concentration within the range of normal human serum levels. Namely, it is preferably added at a concentration of from 30 to 300 U/l . In some cases, it is also added at a concentration in the range of abnormal levels which is usually thrice as high as the normal level and preferably ranges from 120 to 900 U/l.

In the case of using the composition of the present invention as a pharmaceutical composition, it can be administered systemically or topically, and parenterally (preferably intravenously, subcutaneously, percutaneously or intramuscularly) or orally. A CK-heterodimer-containing composition which can be parenterally administered can be prepared within the technical scope in the art by taking pH, isotonicity, safety, etc. into consideration.

The dosage regimen of the composition according to the present invention is appropriately determined by a physician in charge considering the conditions and/or severity of the disease, the body weight and gender of a patient, diet, administration time and other various factors affecting clinical functions. A person skilled in the art can determine the dose of the composition according to the present invention depending on these factors.

If desired, the composition of the present invention can be formulated with physiologically acceptable diluents and/or carriers by various methods. Namely, the composition may be used as a formulation comprising liquid diluents and/or carriers such as an aqueous or oily solution, suspension or emulsion free from pyrogens which can be parenterally administered by injection and which has been sterilized for use therefor as required. It is preferable for the composition of the present invention to be administered parenterally. In the case of oral administration, the composition may contain liquid diluents or carriers. However, such composition will generally contains solid carriers commonly employed in the art, for example, starch, lactose, dextrin or magnesium stearate. The solid composition may be optionally in the form of molded articles such as tablets and capsules including Sapnsule. The composition of the present invention may further comprise auxiliary agents such as preservatives, wetting agents, emulsifiers, dispersants, etc.

The dosage form of the composition thus obtained may be determined depending on the use thereof. Namely, it may be blended with the additives as described above and formulated into tablets, pills, powders, granules, solutions, emulsions and the like.

The composition according to the present invention can be stored over a long period of time without resort to stabilizers.

The invention will now be described in greater detail with reference to the following Examples. However the technical scope of the present invention is not restricted thereto. It is to be understood that modifications and alterations will be apparent to a person skilled in the art depending on the description of the specification without departing from the technical scope of the present invention.

### EXAMPLES

### Example 1

Construction of pTRP Expression Plasmid having CK-M and CK-B Subunit Genes Ligated to Each Other

(1) Isolation of Human CK-M Subunit Gene and B Subunit Gene

By using a commercially available cDNA library (Marathon Ready™ cDNA, manufactured by Clonetech) as a template, PCR was carried out with the use of the following primers. A human CK-M type subunit gene and a human CK-B type subunit gene were thus isolated.

PCR Primers for Amplifying CK-M Subunit Gene

```
PCR primers for amplifying CK-M subunit gene
(Sense primer)
5′-CCGAATTCATGCCATTCGGTAACACCC-3′
        EcoRI site
(Antisense primer)
5′-ATGGATCCCTACTTCTGGGCGGGGATC-3′
      BamHI site
PCR primers for amplifying CK-B subunit gene
(Sense primer)
5′-ATGAATTCATGCCCTTCTCCAACAGCC-3′
        EcoRI site
(Antisense primer)
5′-CCGGATCCTCATTTCTGGGCAGGCATG-3′
      Bam-HI site
```

The sense primer and antisense primer for amplifying the CK-M subunit gene correspond respectively to the nucleic acid sequences 1 to 19 and 1128 to 1143 in SEQ ID NO:2. The sense primer and antisense primer for amplifying the CK-B subunit gene correspond respectively to the nucleic acid sequences 1 to 19 and 1128 to 1143 in SEQ ID NO:4. A restriction enzyme site is added to the 5′-end of each primer so that the nucleic acid fragment can be easily handled. Table 1 summarizes the PCR amplification conditions.

TABLE 1

| | CK-M subunit gene | CK-B subunit gene |
|---|---|---|
| Template CDNA | human skeletal | muscle human brain |
| Polymerase | Taq polymerase | Taq polymerase |
| | (Takara Shuzo) | (Takara Shuzo) |
| PCR reaction | | |
| (Denaturation) | 95° C., 30 sec | 95° C., 30 sec |
| (Annealing) | 55° C., 30 sec | 55° C., 30 sec |
| (Extension) | 72° C., 90 sec | 72° C., 90 sec |
| (Cyce) | 30 | 30 |

The PCR solution employed as a sample was electrophoresed on a 1% agarose gel and thus a PCR product of about 1.2 kbp was confirmed. The 1.2 kbp amplified fragment was purified from the PCR solution by extracting with phenol and precipitating with ethanol and then incubated with restriction enzymes EcoRI/BamHI at 37° C. overnight. The obtained fragment, which had been thus treated with the restriction enzymes, was cleaved from the 1% agarose electrophoretic gel. Next, it was ligated to a pBLuescriptIISK(−) cloning plasmid fragment, which had been subjected to the same restriction enzyme treatment, at 16° C. for 35 minutes by using a commercially available ligation kit (manufactured by Takara Shuzo). The cloning plasmids were named pBluescriptIISK(−)-hCKM and pBluescriptIISK(−)-hCKB respectively and transformed into commercially available E. coli competent cells JM109 (manufactured by Takara Shuzo). The human CK subunit genes thus isolated as E. coli transformants were preserved. Among transformant colonies undergoing blue/white inversion, the plasmid having the CK subunits inserted thereinto was extracted and the gene fragments thus isolated were subjected to DNA sequencing. As a result, the M type subunit gene coincided with the ORF sequence of Genbank Locus HUMCKMA while the B type subunit gene coincided with the ORF sequence of Genbank Locus HUMCKB.

(2) Construction of Plasmid (pTRP-hCKMB) for Expressing Recombinant Human CK-MB Heterodimer Ligated in Tandem

EcoRI/HindIII fragments containing the human CK subunit genes, obtained from the plasmids pBluescriptIISK(−)-hCKM and pBluescriptIISK(−)-hCKB, were each ligated to the EcoRI/HindIII insertion site located downstream of the potent tryptophan promoter of the expression pTRP vector prepared by a method reported by Uchida et al. (Clin. Chem. Acta. 1995, Jun. 15; 237(102):43-58) so that CK-M and CK-B expression plasmids (pTRP-CK-M and pTRP-CK-B) were once constructed.

The following treatments were then carried out so that fragments each consisting of the P promoter, the SD sequence and the CK subunit structural gene were ligated in tandem. First, the HindIII/SalI site (containing trpP and CK-M DNA) was cut out from pTRP-CK-M and inserted into the HindIII/SalI site of the plasmid BluescriptIISK(−). Subsequently, a fragment (containing trpP and CK-M DNA) cut out from BluescriptIISK(−)- trpP+CK-M by using a restriction enzyme XbaI was inserted into the XabaI site located downstream of the CK-B subunit gene in pTRP-CK-B to thereby construct pTRP-CK-M/B.

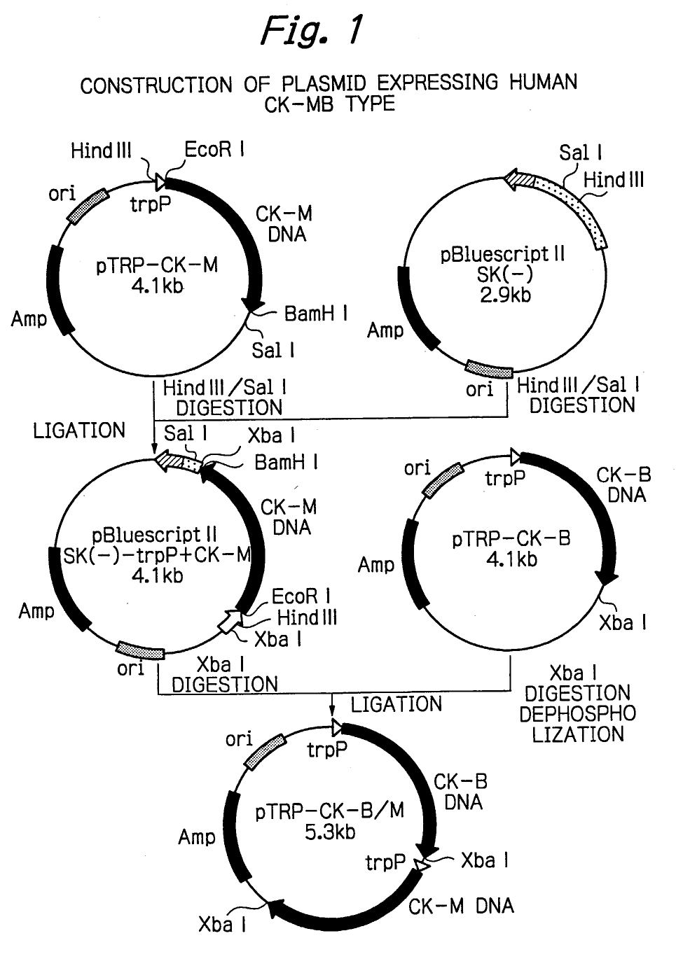

In the pTRP-CK-B/M thus obtained, the TRP promoter, the SD sequence, the CK-B DNA, the TRP promoter, the SD sequence and the CK-M DNA are ligated in tandem. FIG. 1 is a flow chart showing the construction of the plasmid pTRP-hCK-M/B expressing the human CK-MB heterodimer protein.

### Example 2

Separation of Recombinant Creatine Kinase Isozymes

A host strain E. coli JM109 was transformed by using the expression plasmid pTRP-hCK-M/B prepared in Example 1 to give an E. Coli transformant (JM109/pTRP-hCKMB). A process for obtaining an E. coli transformant is described in detail in, for example, “Laboratory Manual for Gene Engineering”, p.109 (ed. Masami Muramatsu, 1988, Maruzen). The transformant JM109/pTRP-hCKMB thus obtained was deposited with National Institute of Bioscience and Human-Technology, Agency of Industrial Science and Technology, MITI (1-1-3, Higashi, Tsukuba-shi, Ibaragi-ken, 305-0046) under the accession number FERM BP-7141 on Apr. 20, 2000.

Next, a human CK expression strain (JM109/pTRP-hCKMB) was cultured under shaking in LB medium (0.5% of yeast extract, 1% of polypeptone, 1% of NaCl) containing 50 μg/ml of ampicillin in a flask at 37° C. overnight.

The cells thus obtained were collected and suspended in a 50 mM phosphate buffer (pH 7.5) containing 5 mM of mercaptoethanol. After disrupting the cells by ultrasonication, the supernatant was referred to as a recombinant CK extract. From this extract, CK was recovered as a precipitate by 70%-saturation ammonium sulfate salting out. The precipitate thus salted out was dissolved in 1.5 M ammonium sulfate (pH 7.5) and subjected to hydrophobic column chromatography by using a Buthyl-Toyopearl C650 gel (manufactured by Tosoh). The CK activity was eluted at 1.5-0 M-gradient (pH 7.5) ammonium sulfate elution. The CK activity elution peaks were combined, concentrated by using an ultrafiltration membrane (YM10, manufactured by Amicon) and then dialyzed against a 50 mM phosphate buffer (pH 7.5). Fractions eluted by the hydrophobic chromatography were adsorbed by a Q-Sepharose column (manufactured by Pharmacia, Sweden) and thus it was attempted to separate CK isozymes.

As a result, the CK activity was separated into the MM type (unadsorbed fraction), the MB type (elution peak 1) and the BB type (elution peak 2). FIG. 2 shows the results. As shown in FIG. 2, the activity ratios of these CK fractions were 45% (MM), 45% (MB) and 10% (BB). It was thus found out that CK-MB was expressed at a high dose in this recombinant strain. It could be also confirmed that three human recombinant CK isozymes could be prepared by using this expression strain alone. The recombinant CK-MB fraction was further subjected to gel filtration chromatography with a Sephacryl-S200HR column (manufactured by Pharmacia, Sweden) and thus purified to electrophoretical homogeneity. The finally purified product showed a specific activity of 533 U/mg.

The CK activity was assayed by using a commercially available human CK activity assay reagent for clinical examination (Merck Liquid-CK, manufactured by Kanto Chemical). The activity assay was performed at 37° C. by using an automatic analyzer (Hitachi Model 7150, manufactured by Hitachi). The unit of enzyme activity was defined as the formation of 1 μmol of ATP per min.

Table 2 summarizes the properties of the recombinant CK obtained in this Example.

TABLE 2

| | |
|---|---|
| CK isozyme expression ratio | MM 45% |
| in the transformant | |
| | MB 45% |
| | BB 10% |
| Subunit molecular weight | 45,000 (SDS-PAGE) |
| Isoelectric point | pI = 5.2-5.3 |
| Specific activity | 533 U/mg (37° C.) |
| Solution stability in serum | Stable after storing at 11° C. |
| | for 120 days or longer |

### Example 3

Solution Stability of Recombinant CK-MB in Defatted Human Serum

The recombinant CK-MB specimen prepared in Example 2 was added to defatted and pooled human serum (CA1, manufactured by Incstar) to give levels of 118 U/l and 449 U/l. These two solutions were allowed to stand in an incubator at 11° C. and stored for 4 months. During this period, the residual CK-MB activity was measured at intervals of 1 month.

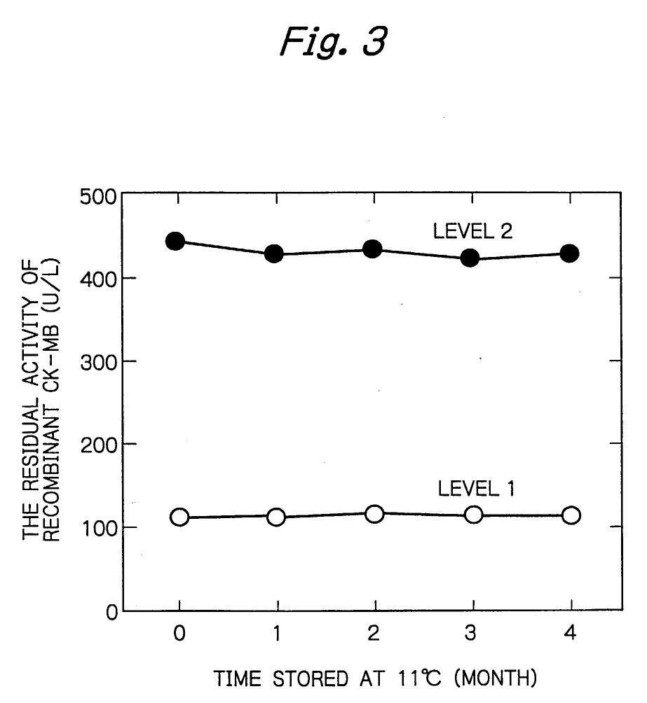

FIG. 3 shows the results. As shown in FIG. 3, the composition according to the present invention at both levels (i.e., 118 U/l and 449 U/l) showed residual activities of 80% or more after storing at 11° C. for 4 months.

### REFERENCES

The following documents are incorporated herein by way of reference.

1. CLINICAL CHEMISTRY, vol. 21, No. 11, p.1691 (1975)

2. Molecular and Cellular Biochemistry 143: pp. 59-65 (1995)

3. CLINICAL CHEMISTRY, vol. 29, NO. 8, pp. 1537-1539 (1983)

4. Japanese Patent Public Disclosure: No.189670/94 (1994. 7. 12)

5. Japanese Patent Public Disclosure: No.252797/97 (1997. 9. 30)

6. Japanese Patent Public Disclosure: No.253378/87 (1987. 11. 5)

7. Japanese Patent Public Disclosure: No.292585/94 (1994. 10. 21)

8. Domestic announcement No.504698/97 (1997. 5. 13) of the PCT International Public Disclosure: WO95/12662

9. “Rinsho Kensa-ho Teiyo (Clinical Examination Handbook)” (31st revised version), pp. 640-643, written by Izumi Kanai, edited by Masamitsu Kanai, Kanahara Shuppan (1998)

10. “Rinsho Kensa-ho Teiyo (Clinical Examination Handbook)”, (31st revised version), pp. 636-640, written by Izumi Kanai, edited by Masamitsu Kanai, Kanahara Shuppan (1998)

11. Perryman, M. B. et al., Biochem. Biophys. Res. Commun., 140(3), pp. 981-989 (1986)

12. Villarreal-Levy, G. et al., Biochem. Biophys. Res. Commun., 144 (3), pp. 1116-1127 (1987)

What is claimed is:

1. A vector comprising a nucleic acid encoding the M type subunit of creatine kinase and a nucleic acid encoding the B type subunit thereof, wherein
i) said M type subunit is a polypeptide having the amino acid residues 1 to 381 in SEQ ID NO:1, or a polypeptide having an amino acid sequence derived from said sequence by deletion, substitution or addition of one or more amino acid residues and having enzymatic activity; and
ii) said B type subunit is a polypeptide having the amino acid residues 1 to 381 in SEQ ID NO:3, or a polypeptide having an amino acid sequence derived from said sequence by deletion, substitution or addition of one or more amino acid residues and having enzymatic activity.

2. The vector according to claim 1 wherein said M type subunit of creatine kinase is a polypeptide having the amino acid residues 1 to 381 in SEQ ID NO:1, and/or said B type subunit of creatine kinase is a polypeptide having the amino acid residues 1 to 381 in SEQ ID NO:3.

3. The vector according to claim 1 wherein said nucleic acid encoding the M type subunit of creatine kinase has the bases 1 to 1143 in SEQ ID NO:2, and/or said nucleic acid encoding the B type subunit of creatine kinase has the bases 1 to 1143 in SEQ ID NO:4.

4. The vector according to claim 3 which is a plasmid.

5. A host cell transformed by a vector as claimed in claim 1.

6. A method for producing a creatine kinase heterodimer recombinant protein containing the M type subunit and the B type subunit of creatine kinase, wherein the method comprises:
i) incubating host cells transformed by a vector as claimed in claim 1 under conditions allowing the expression of the recombinant protein; and
ii) recovering the creatine kinase heterodimer recombinant protein containing the M type subunit and the B type subunit of creatine kinase from the culture medium.

7. A creatine kinase heterodimer recombinant protein containing the M type subunit and the B type subunit of creatine kinase which is produced by the method as claimed in claim 6.

8. A solution-stable composition comprising a creatine kinase heterodimer recombinant protein containing the M type subunit and the B type subunit of creatine kinase.

9. The composition according to claim 8 which sustains its activity at a level of 80% or more after storage at 4° C. for 4 months.

10. The composition according to claim 8 which is free from stabilizers.

11. A composition accoridng to claim 8 which is to be used as a clinical diagnosis control.
